(19) United States
(12) Patent Application Publication
(10) Pub. No.: US 2012/0114639 A1
(43) Pub. Date: May 10, 2012

(54) COMPOSITIONS AND METHODS FOR INHIBITING VIRAL AND/OR BACTERIAL INFECTIONS

(76) Inventors: MICHELLE ADAIR OZBUN, Albuquerque, NM (US); Zurab Surviladze, Albuquerque, NM (US)

(21) Appl. No.: 13/289,220

(22) Filed: Nov. 4, 2011

Related U.S. Application Data
(60) Provisional application No. 61/506,708, filed on Jul. 12, 2011.
(60) Provisional application No. 61/410,136, filed on Nov. 4, 2010.

(51) Int. Cl. A61K 39/395; A61P 31/18; A61P 31/22; A61P 1/16; A61P 31/04; A61P 31/12; C07D 239/94; A61K 31/517; C07D 471/04; A61K 31/519; C07D 311/36; A61K 31/352; C07K 16/46; C12N 5/00; A61P 31/20

(52) U.S. Cl. 424/133.1; 544/293; 514/266.4; 544/279; 514/264.11; 549/403; 514/456; 530/387.3; 435/375

(57) ABSTRACT

We describe herein compositions and methods related to inferring with microbial infection. Generally, the compositions include an infection antagonist that inhibits formation of a heparin sulfonated proteoglycan (HSPG)-containing infection complex. Generally, the methods include administering to a subject an amount of a composition as described herein effective to inhibit infection by a microorganism that that infects a host through interactions that involve HSPG.

### CROSS-REFERENCE TO RELATED APPLICATION

This application claims priority to U.S. Provisional Patent Application Ser. No. 61/410,136, filed Nov. 4, 2010 and U.S. Provisional Patent Application Ser. No. 61/506,708, filed Jul. 12, 2011, each of which is incorporated herein by reference in its entirety.

### GOVERNMENT FUNDING

The present invention was made with government support under Grant Nos. R01 CA132136 and U19 AI084081, awarded by the U.S. National Institutes of Health. The Government has certain rights in this invention.

### BACKGROUND

Human papillomaviruses (HPVs) are small, DNA-containing viruses that infect mucosal and cutaneous epithelium to cause benign and malignant tumors, including many anogenital, oropharyngeal and some skin cancers. HPVs can demonstrate remarkable host restrictions and can have strict tropism for stratifying squamous epithelium. HPV virions consist of 360 copies of the L1 capsid protein, 12-72 copies of the L2 protein and the circular viral genome (≈8 kb) condensed by cellular histones. Like a number of other pathogens, HPV entry into target cells is a multistep process initiated by binding to cell surface attachment factors, the most common of which are glycosaminoglycan chains, especially heparan sulfate in proteoglycans (HSPG). Binding to these negatively charged polysaccharides is usually electrostatic and relatively nonspecific. Many microbes, like HPVs, must transfer from HSPG to a distinct secondary receptor responsible for active pathogen internalization by endocytosis. For HPVs this entry receptor has been elusive.

Despite intensive investigation, the mechanism of HPV movement from primary HSPG attachment receptors to secondary high-affinity receptors has been unclear.

### SUMMARY

In one aspect, the invention provides a composition that has utility inhibiting infection of a subject by an infectious agent. Generally, the composition includes an infection antagonist that inhibits formation of a heparan-sulfonated proteoglycan (HSPG)-containing infection complex. In various embodiments, the infection antagonist can include, for example, a heparanase antagonist, a sheddase antagonist, an inhibitor of an ADAM, an inhibitor of an MMP, an inhibitor of a TIMP, an inhibitor of the release of a growth factor or cytokine, an inhibitor of a molecular association involving HSPG, an inhibitor of molecular associations involving a growth factor receptor (GFR), an antagonist of a sheddase activator, an antagonist of a matrix metalloproteinase (MIMP), an inhibitor of a secretase, an antagonist of growth factor-growth factor receptor (GF-GFR) binding, an antagonist of cytokine-receptor binding, an antagonist of GFR signaling, or an antagonist of receptor-mediated endocytosis.

In some embodiments, the composition can further include a second infection antagonist.

In another aspect, the invention provides a method that generally includes administering to a subject a composition comprising an infection antagonist that inhibits formation of a heparin sulfate proteoglycan (HSPG)-containing infection complex in an amount effective to inhibit infection by a microorganism that that infects a host through interactions that involve HSPG.

In some embodiments, the microorganism can be a virus. In other embodiments, the microorganism can be a bacterium.

### BRIEF DESCRIPTION OF THE DRAWINGS

The patent or application file contains at least one drawing executed in color. Copies of this patent with color drawing(s) will be provided by the Office upon request and payment of the necessary fee.

FIG. 1. HPV16 and HPV31 interact with HSPG and syndecan-1 at the plasma membrane of HaCaT cells. (A-D) HaCaT cells were exposed to HPV16 or HPV31 virus particles at 4° C. for one hour, and then washed. Cells were either fixed for confocal fluorescence microscopy or were lysed in RIPA buffer (E). HaCaT cells were immunostained for HSPG (A and C) or syndecan-1 (B and D) and HPV16 (A and B) or HPV31 (C and D). (E) The following samples were analyzed by SDS-PAGE and immunoblot with anti-syndecan-1 antibody: lane 1) Magnetic beads and anti-HPV16; 2) IP of HPV16 from HaCaT cell lysate; 3) IP of HPV16 from HaCaT cells treated with DTSSP before lysis; 4) blank lane; and 5) HaCaT cells lysate (no antisera).

FIG. 2. HPV16 and syndecan-1 release from the HaCaT cell plasma membrane is MMP dependent. (A, B) Immunoblot for syndecan-1 (polyclonal syndecan-1 Ab) after 24 hours at 37° C. in CM (A) or post starvation in SFM and after six hours at 37° C. in Tyrode's Buffer (B); immunoblot for HPV16 L1 released into CM post virus binding for indicated times (C) or 24 hours (E). (D) Gelatin zymography showing protease activity present in HaCaT cell CM alone or with binding of HPV16. (E-F) Effect of MMP inhibitors batimistat (BM) or marimastat (MM) at the indicated concentrations on HPV16 release into media as in panel C and densitometric quantification with AlphaEaseFC software (E) and relative infection levels (F). Cells were untreated (U) or pre-treated with BM or MM for one hour, then exposed to HPV16 PsV in the presence of inhibitors in CM. Infection was assayed by quantifying luciferase levels at 24 hours p.i. (F). Data are represented as mean±SEM of three experiments. (G-H) IC₅₀ of HPV16 infectivity inhibition by MMIP inhibitors batimastat and mariniastat. HaCaT cells incubated with serial dilutions of batimastat or marimastat in CM, for one hour before incubation with 100 viral genome equivalents (vge)/cell HPV16, one hour at 4° C. After washing away unbound virus, cells were incubated for 24 hours at 37° C. in the presence of inhibitors. HPV16 infection was measured with luciferase assay. Data are represented as mean±SEM of 3 experiments.

FIG. 3. Sepharose 4B gel chromatography of media constituents from HPV-exposed HaCaT cells. Sepharose 4B chromatography was performed on released components in the CM of HaCaT cells exposed to HPV16 PsV for four hours. (A) The void volume (HMW, FIG. 3D, lane 1) fraction was divided into four parts that were untreated or incubated with 1 U heparanase III for two hours at 37° C. then solubilized in 6× sample buffer and incubated at 25° C. or boiled (95° C. in Albuquerque, N.Mex.) for seven minutes before SDS-PAGE and L1-immunoblot analysis. (B) Non-reducing SDS-PAGE of void volume Sepharose 4B fractions from released components in the CM of HaCaT cells mock-exposed or HPV16-exposed for 24 hours. Immunoblot analysis was done to detect L1, amphiregulin (AREG), BB-EGF, EGF, HS and syndecan-1. (C) Non-reducing SDS-PAGE of released components following IP of HPV16 from CM of HaCaT cells mock-exposed or HPV16 -exposed for six hours or 24 hours. Immunoblot analysis was done to detect HB-EGF, EGF, and syndecan-1. (D-E) Eluted fractions from Sepharose 4B column chromatography (indicated at top of gels) were solubilized in 6× sample buffer, boiled for three minutes (95° C. in Albuquerque, N.Mex.). Samples were separated by 10% SDS-PAGE followed by electrotransfer. Lane numbers are indicated below each blot. Membranes were probed for (D) HPV16 L1 using mouse mAb and (E) for syndecan-1 using rabbit antisera. Lane 11 in Panel E contains HaCaT cell lysate as a control.

FIG. 4. Released HMW complexes including HSPG and HPV16 are required for infection. (A-D) Schematic of the “donor” cell/“recipient” cell co-culture system indicating how cells were PsV exposed. PsVs were allowed to bind donor cells without internalization at 4° C. (A). Donor cells on coverslips were washed thoroughly to remove unbound PsVs and transferred to mesh inserts above the recipient cells to co-culture with gentle rocking for 24 hours and allow released HPV complexes from donors to access the recipient cells (C-D). All experiments employed CM. (E) Relative HPV16 infection levels of HaCaT donor cells and recipient HaCaT cells compared to mock infected cells as verification of the co-culture virus release model. (F) IP was performed by immobilizing anti-L1 in the lower chamber in place of cells (FIG. 4B) to capture HPV16 released from mock exposed cells (M) or HPV 16-exposed HaCaT donor cells at two or 20 hours post virus exposure. Lower panel IgG detection is included as a loading control. (G) Relative infection levels in CHO-K1 and pgsd-677 cells used as donor cells bound to HPV16 PsVs and co-cultured above the recipient cells. (H) Relative infection levels in CHO-K1 and pgsd-677 cells used as recipient cells co-cultured below the PsV-bound donor cells corresponding to the data in panel G. Infectivity data (E,G,H) were normalized to the mean value of the infected control set to 100% and represent the mean±SEM of four replicate infections.

FIG. 5. HPVs interact with growth factors and growth factor receptors on human keratinocytes. Immunofluorescent confocal co-localization (arrowheads show examples of signal overlap) showing top view and side views of non-permeabilized HaCaT cells. (A) Co-localization of HPV16 (red) with EGF (green). Bars measure 10 μm. (B) Co-localization of HPV16 (green) with KGFR (red). Bars=5 μm. (C) SDS-PAGE and immunoblot for EGFR or p-KGFR after IP of I-IPV16 from HaCaT cells exposed to PsV. Lane 1, HaCaT cell lysate (without HPV exposure) incubated with anti HPV antibody attached to magnetic beads (negative control); lane 2, IP of HPV16 (mouse monoclonal anti-L1) from HaCaT cells; lane 3, blank; lane 4, HaCaT cell lysate following EGF exposure as positive control.

FIG. 6. HPV16 activates EGFR and KGFR signaling pathways. (A-B) Immunoblot of p-EGFR, p-KGFR and downstream effector p-ERK1/2 following 10 minutes ligand exposure (listed at the left of each blot: EGF, KGF, HPV16 PsV; lane 2) in serum-starved HaCaT cells. Mock exposed cells were negative controls (lane 1); ligand controls included 10 ng/ml EGF and 10 ng/ml KGF exposure (10 minutes). Cells were pretreated with the indicated inhibitors (100 nM PD168393, 1 μM PD173074, 100 μM genistein, 100 μM daidzein, 600 nM cetuximab) and exposed to the indicated ligands (HPV16, EGF, KGF) for 10 minutes in the presence of inhibitors. Actin is detected as a loading control. (C-D) Immunoblot of nuclear cell fractions and confocal microscopy localization of p-ERK1/2 following EGF and HPV16 exposure in serum-starved HaCaT cells at indicated times post-exposure. (C) Immunoblot for p-ERK1/2 in nuclear fractions from exposed cells. (D) Immunofluorescent confocal microscopy for localization of p-ERK1/2 (red) in PsV exposed cells. DAPI was used as a nuclear marker and was pseudocolored green to facilitate efficient co-localization of p-ERK1/2 in the nucleus. Parameters of lasers intensities were kept constant during the imaging.

FIG. 7. GFR activation, serum components including GF, and EGFR are important for HPV16 infections. (A) Relative HPV16 infection of HaCaT cells in the presence of specific GFR and protein tyrosine kinase inhibitors. Subconfluent HaCaT cells were pre-treated 45 minutes with 1 mM AG1478, 100 nM PD168393, 100 μM genistein, 100 μM daidzein, 1 μM PD173074, 100-600 nM cetuximab. Cells were exposed to HPV16 PsV at 100 vge/cell for one hour at 4° C., then washed extensively and shifted to 37° C. in the presence of the indicated inhibitor in CM for 24 hours at which time they were analyzed for luciferase expression. Data are represented as mean±SEM of three experiments. (B-C) EGFR knockdown was accomplished using siRNA to EGFR and the percent of EGFR protein remaining in EGFR-siRNA transfected HaCaT cells was determined by immunoblot and compared by densitometry to EGFR levels in cells transfected with a negative control siRNA at 48 hours post transfection. Four separate transfections were analyzed (B) and HPV16 PsV infection levels were measured at 24 hours post infection (48 hours post transfection) in each (C). Error bars represent the average of triplicate luciferase readings from the four transfections. (D) Cells were pre-treated four hours with 100 μm monastrol, pre-treated with monastrol for four hours plus PD168393 for duration, or pre-treated with PD168393 for four hours plus monastrol for duration. (E) Relative HPV16 infection is dependent upon medium constituents post primary HPV16 binding. HaCaT cells starved in SFM (four hours) were exposed to HPV16 in CM (positive control) or SFM. After washing away unbound virus, cells were incubated for 24 hours in CM, Ty (Tyrode's buffer containing 0.05% BSA), SFM, syndecan-1 depleted CM (IP-syd), or EGF-depleted CM (IP-EGF). Infection was quantified by luciferase assay 24 hours post shift to 37° C. Data are represented as mean SEM of three experiments. (F) Relative HPV16 infection in SFM is enhanced by GF. HaCaT cells starved in SFM were exposed to HPV16 in SFM for one hour at 4° C. After washing away unbound virus, cells were incubated for 24 hours in. SFM, SFM containing GF, or in CM. Infections were quantified by luciferase assay; bars represented the mean SEM of three individual experiments. (G). To investigate if infection inhibition by the various agents used in FIG. 7A,D,E could be attributed to cell cycle changes, we used identical conditions and timing of inhibitor treatment on HaCaT cells. We then stained with propidium iodide and examined the cell cycle using flow cytometry. The fractions of cells in G1 (1n), S (intermediate), and G2/M (2n) phases were expressed as percentages of the total.

FIG. 8. Model for extracellular interactions of HPV in the context of normal HSPG biology. (A) Natural processes that occur in the absence of HPV. The edges of epithelial cell lipid bilayers are depicted interacting with the extracellular matrix (ECM). Laminin 332 (formerly laminin 5) interacts with syndecane-1. and alpha-6 beta-4 integrin on the cell surface to provide cell anchorage to the ECM/basement membrane. These three molecules have been proposed as HPV attachment factors. Matrix metalloproteinases (MMPs) and ADAM sheddases normally catalyze the release or “shedding” of membrane-bound growth factors (GF) and other bioactive molecules (i), the protein ectodomains of heparan sulfonated proteoglycans (HSPG) like syndecan-1, and ECM residents like laminin 332 (dotted arrow). The ECM can act as a reservoir for GF and bioactive molecules (ii.) HSPG in the plasma membrane and ECM act as local depots for soluble GF and other bioactive molecules (ii). The GF and bioactive compounds can interact with their cognate receptors laterally, via soluble form after release, or in the ECM when cells migrate over the HSPG-complexes (iii.) Soluble complexes containing GF and HS±syndecan-1 are liberated by heparanases and proteolytic processing of laminin 332 (iv) Soluble GF complexes bind to GFR/RTK and activate intracellular signaling cascades. (B). The natural processes of HSPG decoration and release from the cells also occurs in the presence of HPVs. By virtue of interaction with HS, HPV can join the complex at each stage where HSPG is involved (i-iv). HPV could associate with soluble HS-GF in a naïve infection site or during release from infected cells (v). HPV16 association with synedcan-1 via HSPG and binding of syndecan-1 to laminin 332 and alpha-6 beta-4 integrin are consistent with the fact that HPV particles co-localize and interact with each of these extracellular molecules. The abundance of HSPG in the ECM can explain why HPVs bind at such high levels to the ECM (ii). Cells can pick up HPV-HS-GF complexes in soluble form or by migrating over ECM-bound HSPG-GF (HPV) complexes.

### DETAILED DESCRIPTION OF EXEMPLARY EMBODIMENTS

Heparan-sulfonated proteoglycan (HSPG) interactions are involved in many infections including, for example, infections by human papillomavirus (HPV). In the description that follows, HPV is used as an exemplary model infectious agent. This disclosure, however, shall not be construed as limited to infection by HPV, as the mechanisms described with reference to HPV are not specific to HPV infection and therefore correspond to mechanisms involved in infection by other infectious agents.

HSPG can activate a conformational change in the virus that allows engagement with the entry receptor. Thus, in the HPV example, the virus may bind to HSPG (e.g., syndecan-1) and this HPV-HSPG complex can, along with growth factors (GFs) or other bioactive HSPG-binding proteins, be shed (or released) from the extracellular matrix or plasma membrane Inhibiting this shedding or release of the HPV-HSPG complex can inhibit infection. A released complex that includes the virus and HSPG (e.g., syndecan-1) and growth factors (or other bioactive molecules) can cause infection. We find that growth factors are required to interact with HPV and HSPG in an “infectious complex.”

Furin also can be involved in mechanisms of infection by cleaving, for example, the HPV minor capsid protein L2. Furin also can cleave and thereby activate matrix metalloproteinases (MMPs) that can be involved in shedding of HSPG (e.g., syndecan-1) and shedding of epidermal growth factor (EGF)-family and fibroblast growth factor family of growth factors from their transmembrane domains. Although furin can increase virus release from cells slightly, virus bound and exposed to furin in the absence of growth factors typically are not infectious. Thus, furin also may be involved in infection by activating cellular ADAM (i.e., a disintegrin and metalloprotease) sheddases, which can release growth factors and HSPG from cells. Carrageenan can inhibit infection by blocking interactions between the cell-surface HSPG attachment factors and, for example, the viral capsid. An HSPG-independent inhibitory effect was athibuted to occlusion of virion surfaces or interfering with the purported L1 conformational changes involved in secondary receptor binding. Carrageenan also may be involved in absconding with growth factors, which are commonly involved in, for example, virus internalization and infection.

Secretases can inhibit infection by altering L2. Secretases can be involved in the release of HSPG/GF and/or, for example, HPV infectious complexes from cells.

Infectious agents such as, for example, oncogenic HPVs, can physically associate with both HSPG—predominantly syndecan-1—and bioactive compounds like growth factors to form a high molecular weight (HMW) association called an “infectious complex” in order to interact with their “secondary” and internalization receptors. Many infectious complexes or constituents thereof require release or “shedding” from the plasma membrane or extracellular matrix of keratinocytes before these HMW complexes can efficiently interact with the secondary receptors.

Internalization receptors include growth factor receptors like epidermal growth factor receptor (EGFR) and fibroblast growth factor 2IIIb (FGFR2IIIb, also sometimes referred to as keratinocyte growth factor receptor, KGFR). The HPV/HSPG/GF receptor interaction and signal transduction that results from the engagement are typically involved in HPV uptake and can be involved in preparing the cell to receive and express the viral genome in its nucleus.

Thus, in one aspect the invention relates to compositions that include an infection antagonist. As used herein, the term “infection antagonist” refers to a molecule that inhibits one or more aspects of the infection pathway discussed above so that the extent, severity, frequency, and/or likelihood of infection by a microorganism that infects a host via the pathway described herein is reduced. The extent, severity, frequency, and/or likelihood of infection by such a microorganism can be routinely monitored by assessing the extent, severity, frequency, and or likelihood of a symptom or clinical sign that is characteristic of a condition caused by such an infection compared to an appropriate control. Exemplary aspects of the infection pathway include, for example, GF/HSPG association, GF/GFR association, and/or GFR signaling.

The antagonists listed below can be viable candidates to inhibit infection by HPVs. Further, these antagonists can inhibit binding and uptake of other microorganisms—whether viral or bacterial—that involve HSPG interactions prior to cellular uptake such as, for example, retroviruses (e.g., HIV), herpes simplex viruses or other herpeseviruses, hepatitis C virus, Vaccinia virus, Chlamydia, and Neisseria. The infection antagonists can include an inhibitor of a heparanase, an inhibitor of a sheddase (e.g., a disintegrin and metalloprotease, ADAM), an inhibitor of the maturation of or release of a growth factor (GF), an inhibitor of molecular associations involving HSPG, an inhibitor of molecular associations involving a GFR, an antagonist of a sheddase activator, an antagonist of a MMP (e.g., a TIMIP3 or furin inhibitor), an inhibitor of a secretase, an antagonist of GFR-GF binding, an antagonist of GFR signaling, or any combination of two or more of the foregoing antagonists. Infection antagonist can inhibit infection by viruses such as, for example, HPV, HIV, and herpes simplex. Infection antagonists can inhibit infection by bacteria such as, for example, members of the genus Chlamydia such as, for example, C. trachomatis, C. suis, and C. muridarum, or members of the genus Neisseria such as, for example, N. gonorrhoeae.

Accordingly, the invention also involves methods of inhibiting infection in a subject by administering to the subject an amount of a composition effective to inhibit infection by a microorganism that infects a host through interactions that involve HSPG.

Glycosaminoglycans (GAGs), including HSPGs, are expressed on the surface of nearly all cells linked to transmembrane proteins. These GAGs can be sulfated to varying extents, lending a negative charge, and are used by a large number of microorganisms to initiate infection. Heparin sulfonation can provide an initial docking site at the plasma membrane that then permits subsequent interaction with a specific receptor. Syndecan-1, an abundant HSPG in keratinocytes, is an exemplary attachment receptor for oncogenic HPVs.

A prominent characteristic of syndecans is that their extracellular domains can be cleaved to release intact HS ectodomains decorated with bioactive molecules that act as soluble effectors. All syndecan ectodomains are shed constitutively as a normal part of turnover, but this process is also regulated (e.g., certain GF accelerate shedding). The enzymes responsible for syndecan shedding are the matrix metalloproteinase peptidases (MMPs) that cleave the syndecan core protein and release the ectodomains. The HS moieties on syndecans also can be degraded by heparinases, which can liberate the HS bound to GF and bioactive compounds. HPVs can attach to HSPG on the extracellular matrix (ECM) and the plasma membrane of human keratinocytes. The virus, particles may undergo a conformational change, and the HPV-HSPG complex may then be transferred to an uptake receptor to facilitate the infectious process. We investigated the mechanism by which HPVs are transferred from the primary HSPG attachment to the internalization receptors.

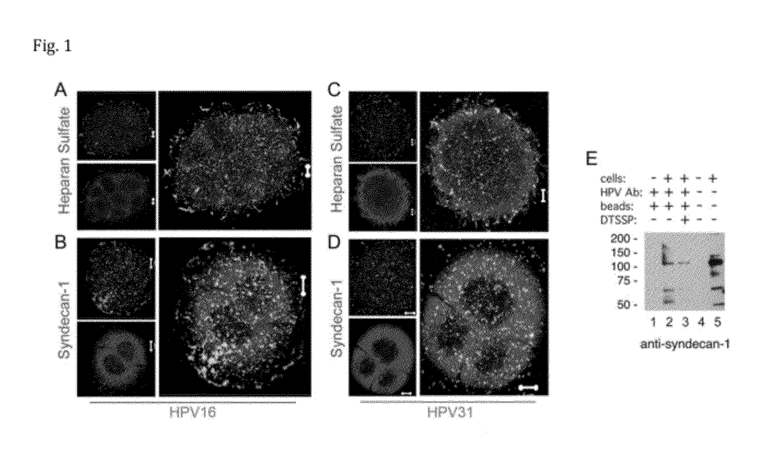

We performed an assay to determine the association of virus particles with HSPG and syndecan-1 on human keratinocytes (HaCaT), a permissive cell for oncogenic HPV16 and EIPV31. FIG. 1 shows HaCaT cells exposed to HPV16 or HPV31 virus particles. Confocal microscopy was performed following immunostaining for the indicated viruses and either the HSPG (HS) or syndecan-1 (Snd-1). The yellow spots are indicative of virus co-localization with HS and syndecane-1. . In the panel to the right, HaCaT cells were exposed to HPV16 particles and washed to remove unbound particles. Cell lysates were prepared and immunoprecipitated with affinity purified rabbit anti-HPV16 VLP antisera. The samples were analyzed by SDS-PAGE and then immunoblotted with anti-syndecan-1 antibody. The data together demonstrate that HPV16 and HPV31 particles interact with syndecan-1 specifically.

MMPs Contribute to Cell Surface Release of HPV, which is Important for Infection

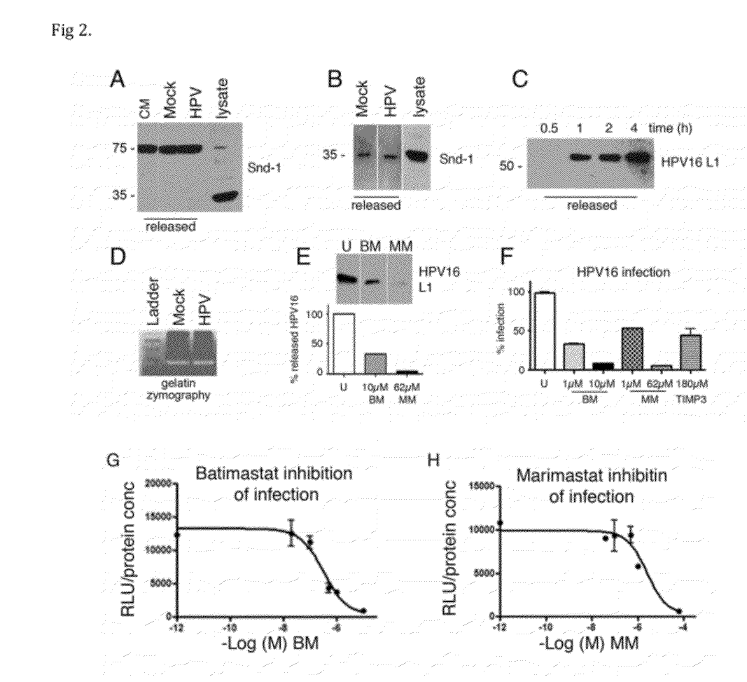

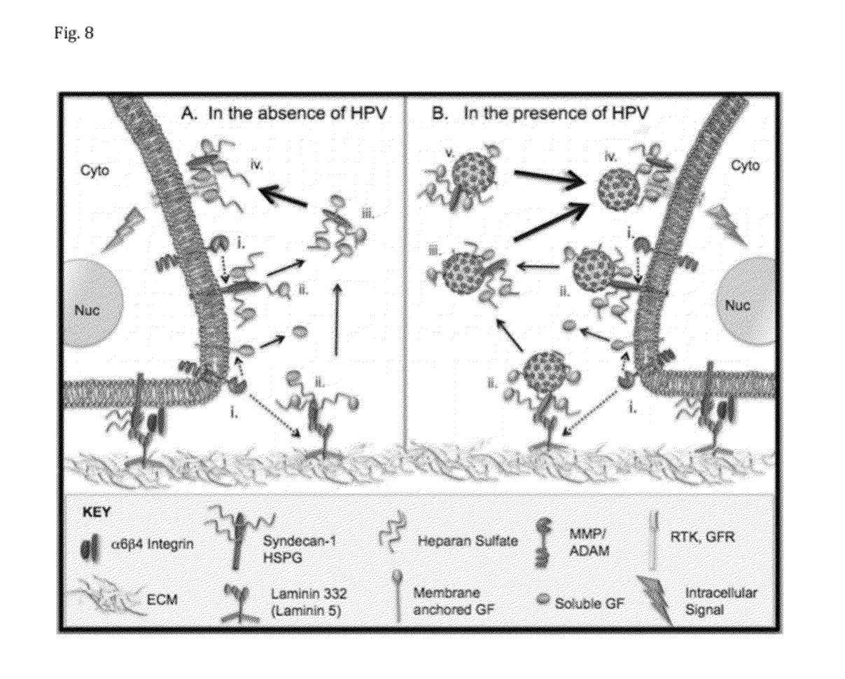

To verify HPV particles bind to syndecane-1 on HaCaT human keratinocytes, we used confocal microscopy and immunoprecipitation (IP) (FIG. 1). HSPG including syndecans-1 are actively shed from epithelial cells via the activity of a variety of MMPs including, but not limited to, MMP7, MMP9, MT1MMP, ADMTS1, ADAM17, and LasA (FIG. 8A). To test whether HSPG-bound HPV particles are released from cells in complex with HSPG and syndecans, we collected media from HaCaT cells growing in complete medium (CM; DMEM containing 10% FCS), including from HPV16 PsV-bound cells, and assayed for released syndecan-1 and HPV. Immunoblot analysis for syndecan-1 showed that CM itself contained substantial levels of what appeared to be a dimeric form of syndecan-1 (FIG. 2A). This finding complicated the determination of virus binding effect on syndecan-1 release. To resolve the problem of free syndecan-1 in CM, we instead used serum-free medium (SFM) or Tyrode's buffer solution. Cells starved in SFM were exposed to HPV16 at 4° C. and analyzed for syndecan-1 release after maintenance for six hours in Tyrode's buffer at 37° C. Immunoblot showed that cells released the ˜35 kDa ectodomain form of syndecan-1 in the absence and presence of HPV16 (FIG. 2B). HPV did not significantly accelerate syndecan shedding as reported for several bacterial pathogens.

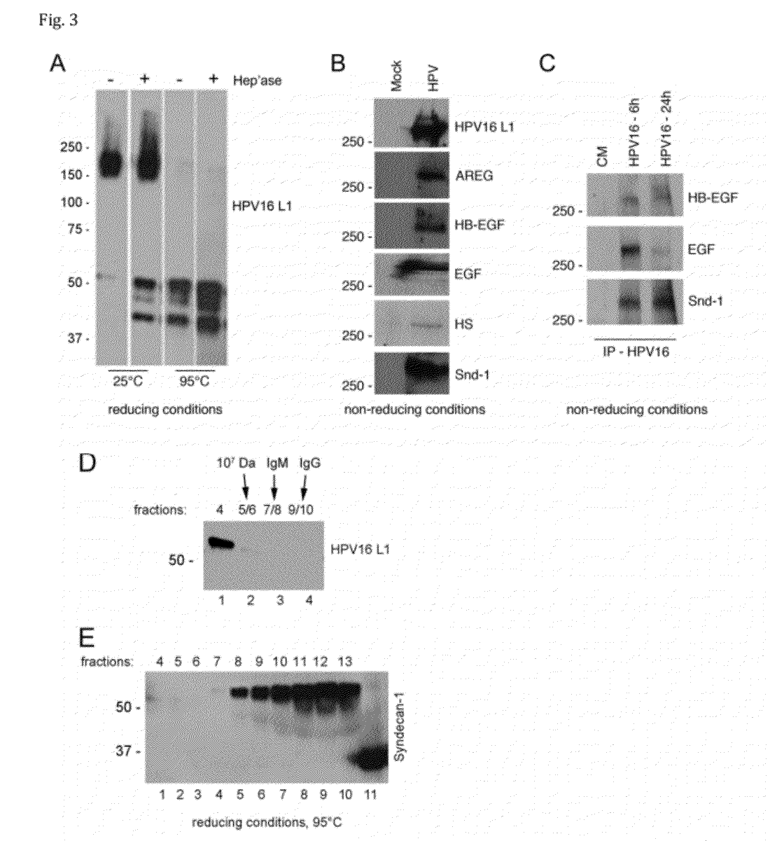

To characterize HPV16 released from cells, media from virus-exposed cells were concentrated with Amicon 30 ultrafilters then applied on a Sepharose 4B column. This method is used widely in cell biology to isolate and characterize differently sized complexes. Size-exclusion chromatography fractions were analyzed by SDS-PAGE and immunoblot. HPV16 eluted in void volume fractions of this highly porous gel—fraction 4 contains large complexes or particles with MW >10⁷ Da) (FIG. 3D, E). Immunoblot analysis of void volume fractions revealed that the amount of HPV released into the medium increased with time (FIG. 2C).

There are at least two explanations for release of bound HPV16 from the cell surface in CM. First, the non-covalent association of HPV to HSPG is dynamic and viral particles could dissociate from the cell and associate with soluble syndecan-1 in the serum-containing CM. The high concentration of HS in serum could compete for virus binding to the cell. Second, HPV could be released in complex with syndecane-1 or HS via the activity of MMP cleaving the anchored ectodomain of the HSPGs or by heparinases liberating HS. The second scenario is consistent with our finding that syndecane-1. (FIG. 2B) and HPV16 are released in Tyrode's buffer.

Gelatin zymography analysis revealed the presence of gelatinases in experimental medium (FIG. 2D). However, this sensitive and widely used method detects non-active latent MMP forms in addition to active forms. Therefore, to more specifically test the involvement of MMP activity in HPV release and infection, we investigated the effects of MMP inhibitors on virus release and infection. The large number of MMPs important for epithelial cell HSPG ectodomain shedding and fact that many possess overlapping substrates in vitro make genetic knockdowns unfeasible. Since we wished simply to determine if HSPG release was related to HPV16 infectivity, we tested broadly active MMP inhibitors, batimastat (BM) and marimastat (MM), which are typically used to assay the functional consequences of inhibiting MMP activity and the release of their substrates. These widely used hydroxamic acid MMP inhibitors are well known to block the release of syndecans from cells. Both BM and MM effectively prevented the release of HPV16 from cells (FIG. 2E) and efficiently reduced HPV16 infection of HaCaT cells (FIG. 2F). TIMP3 blocks MMP activity, can prevent syndecane-1 release, and also can prevent HPV16 infection (FIG. 2E).

Our dose-response analysis of batimastat and marimastat revealed HPV16 infection inhibition at an IC₅₀ of 400 nM (BM) and 1 μM (MM) in the absence of visible toxicity (FIG. 2G, H). Thus, the actions of the inhibitors indicate that MMPs are involved the release of virus from cell membranes and that virus release plays an important role in infection.

HPV Particles Released in HMW Complexes are Associated with Syndecan-1, HS and Growth Factors

Syndecan HSPGs can participate in assembling signaling complexes by, for example, accumulating biological mediators including GF and presenting these factors to their high affinity receptors. Therefore, released HPV particles can be in complex with HS (or HSPG) of varied sizes along with assorted GFs. Solubilization of the Sepharose 4B void volume fraction in SDS-mercaptoethanol sample buffer and boiling caused complete dissociation of virus resulting a single monomeric ˜55 kDa band of HPV16 L1 protein (FIG. 2C). We found that viral complex dissociation can be influenced by temperature. For example, without heating, HPV16 L1 in SDS-reducing buffer was detected only in a form >150 kDa (FIG. 3A). These results indicate the cell surface-released HPV is part of a detergent-resistant and temperature-sensitive HMW complex.

To determine the role of HS in this complex, the Sepharose 4B void volume fraction (MW >10⁷ Da) was exposed to heparanase III. Treatment with heparanase III induced partial dissociation of HMW complexes and a considerable amount of soluble HPV16 L1 was detected at ˜55 kDa, indicating that HS is involved information of HMW virus-containing complexes. Under non-reducing conditions in BMW fractions, HPV16 L1 appeared as >250-kDa (FIG. 3B), suggesting that the reducing conditions caused dissociation of some complexes. This is in contrast to the fact that L1 proteins from purified mature HPV PsVs appear as 125 kDa dimers and 195 and 215 kDa trimers under non-reducing SDS-PAGE conditions, but never migrate above 215 kDa.

Next we used the HMW void volume Sepharose 4B fraction for analysis of GF and HS. Individually these molecules are low molecular weight and mainly elute from the column in later fractions (>9, FIG. 3E). Fractionated media from mock exposed HaCaT cells was a control. Immunoblot analysis revealed the presence of amphiregulin (AREG), heparin binding epidermal growth factor (HB-EGF), EGF, HS, and syndecane-1 but only in HMW fractions of media from cells exposed to HPV16 (FIG. 3B). Non-reducing SDS-PAGE of this void volume Sepharose 4B fraction shows all of these molecules are present, each appearing to be >250 kDa in size. To more specifically assess the direct association of these components, we performed an immunoprecipitation for HPV16 particles released into CM following virus binding to cells at 4° C. and shift to 37° C. for six or 24 hours. Immunoblotting for HB-EGF, EGF, and syndecan-1 demonstrated these factors were in a soluble complex with HPV16 released from cells (FIG. 3C). These findings suggest HPV particles released in HMW complexes from cells are “decorated” with syndecan-1 ectodomains, HS, and assorted GF. To our knowledge this is the first demonstration of an attached incoming non-enveloped virus being liberated from the cell surface into the experimental medium. Further, this is the first report of a mechanism, distinct from dissociation, by which the virions are released from cells.

### Released HPV16 Complexes are Infectious and HSPG Play a CrucialRole in Infection

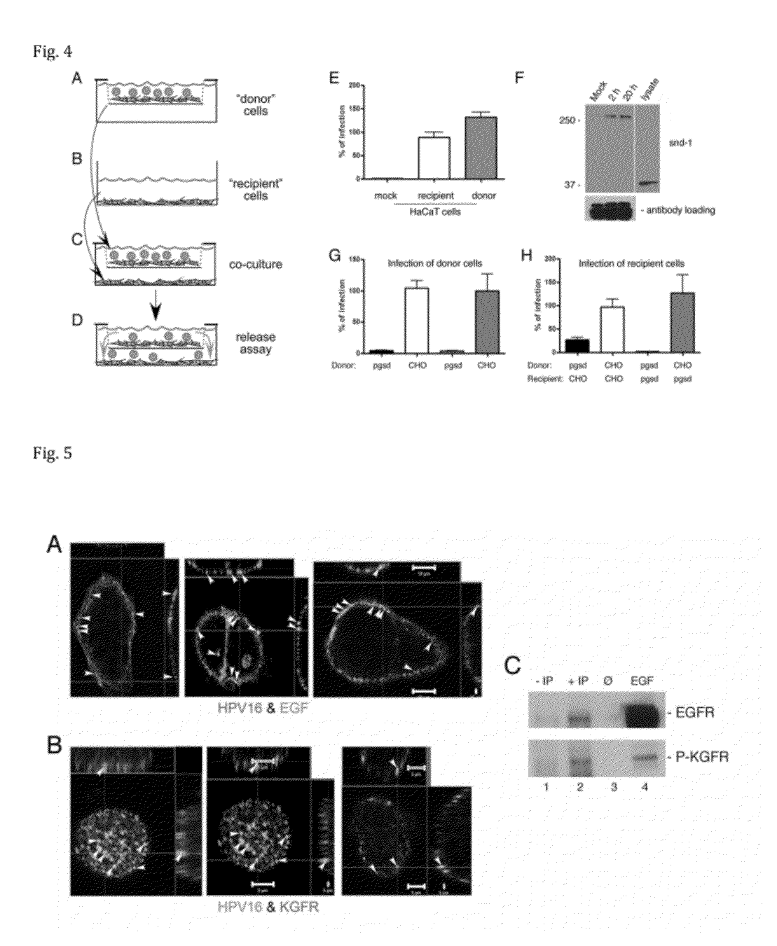

To ascertain if released virus complexes were infectious, we designed a co-culture transwell system wherein unexposed (“recipient”) cells were cultured in chambers below an insert holding “donor” cells that separately had been exposed to HPV16 (FIG. 4A-D). As a proof-of-principle, HaCaT cells were tested as both donor cells and recipient cells (FIG. 4E). HaCaT donor cells were allowed to bind HPV16, washed to remove unbound virus and placed atop recipient HaCaT cells where they were incubated with gentle rocking for 24 hours. Comparable infection levels were detected between directly PsV-exposed HaCaT donor cells and the recipient HaCaT cells grown in the lower chamber demonstrating the infectivity of the released HPV16 material (FIG. 4E).

To verify that HPV16 released from donor cells was in a complex with syndecan-1, we used bead-attached anti-HPV16 antibody instead of recipient cells in the lower chamber. Following capture of the viral particles, non-reducing SDS-PAGE and immunoblot with anti-syndecan antibody confirmed the co-immunoprecipitation of syndecan with released HPV16 (FIG. 4F). Similar to when the material released into cell media was subjected to chromatography (FIG. 3), the syndecan-1 plus L1 complex released from donor cells appeared as a HMW form >250 kDa. Conversely, only the 35-kDa monomeric form of syndecane-1 was detected in the cell lysate from cells not exposed to HPV (FIG. 4F).

To determine the importance of HS in the infectious process following PsV release, we tested wild-type Chinese hamster ovary (CHO-K1) cells and mutant CHO cells defective in HS biosynthesis (pgsd-677). We found the HSPG-defective cells could be infected by HPV16 PsV, but at levels reduced to only ≈5-8% of the wild-type CHO cells (FIG. 4G). Using our co-culture system, PsV-exposed CHO-K1 or pgsd-677 donor cells were placed atop of CHO-K1 or pgsd-677 cells grown as recipient cultures. Infections were assayed in respective donor and recipient cells from these co-cultures (FIGS. 4G and 4H, respectively). Donor CHO-K1 cells exposed to HPV16 PsV could fully confer infection to recipient CHO-K1 cells (FIG. 4H, CHO/CHO). Importantly, recipient pgsd-677 cells were also fully able to support infection, but only when CHO-K1 cells were used as PsV donors (FIG. 4H, CHO/pgsd). These results demonstrate for the first time that HSPG attachment receptors are not required for recipient cell infection when HPV particles are released in complex with HSPG from donor cells that are able to express HSPG. These data show an infectious role for the released HMW complexes containing HPV16 decorated with HS on cells that lack HSPG. That donor pgsd-677 cells could confer limited infection to CHO-K1 cells (FIG. 4H, pgsd/CHO) may reflect low level dissociation or release of virus to the fully receptive HSPG-wild type CHO-K1 cells.

Growth Factors Present in BMW Complexes with HPV Facilitate Growth Factor Receptor Interactions

The very high affinity of GFs for their specific receptors (K _{D}≈10-100 pM) may permit the GFs to influence the fate of the virus-cell interaction prior to HPV entry. Detecting interaction of virus with GF receptors (GFR) is an indication of this possibility. Thus, co-localization of HPV16 with GF and GFR was assayed by confocal microscopy and physical associations were tested by co-immunoprecipitation. HaCaT cells exposed to HPV16 were either incubated with fluor-labeled EGF or immunostained for FGFR2IIIb/KGFR. FIG. 5A and FIG. 5B show the partial co-localization of HPV16 with EGF and KGFR on the cell plasma membrane. Immunoprecipitation of HPV16 PsV provided additional evidence of interactions with EGFR and KGFR following PsV binding to HaCaT cells. Immunoblot analysis demonstrated the co-immunoprecipitation of EGFR and phospho-KGFR from HaCaT cells following the immunoprecipitation of HPV16 PsV (FIG. 5C). These data confirm the interaction of HPV16 with EGFR and KGFR on the plasma membrane of human keratinocytes. The chromatography and immunoprecipitation data together support the idea that HPV particles become decorated with HS and bioactive molecules like GFs such as, for example, EGF and KGF (FIG. 3B-C) to interact with GFRs.

### HPV Exposure Induces Rapid GFR Phosphorylation and Activation of Downstream Effectors

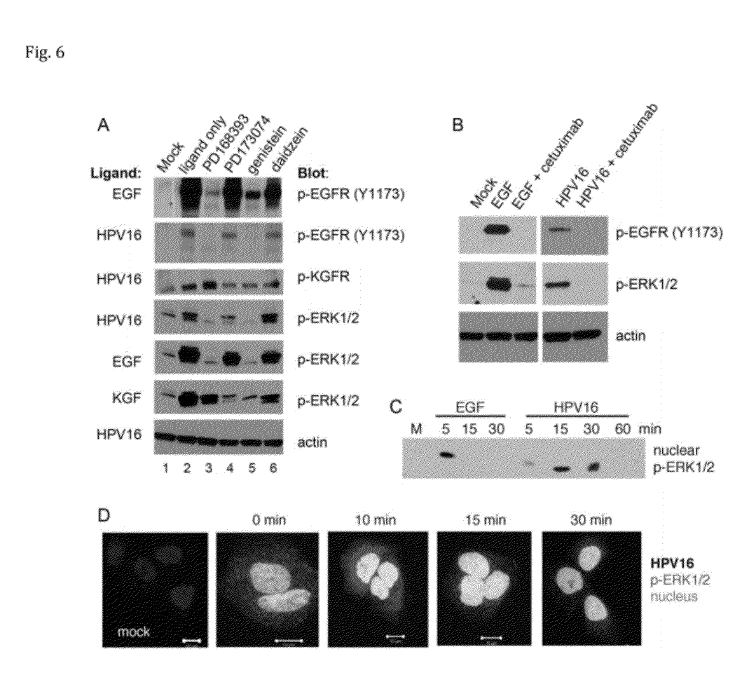

The engagement of GFR by their ligands induces rapid auto-phosphorylation and downstream signaling. To investigate the involvement of EGFR and KGFR activation and signaling in HPV infections, we analyzed phosphorylation levels of the GFR and mitogen-activated protein kinases (MAPK) ERK1/2. HaCaT cells starved in SFM for four hours were incubated with low doses of HPV PsV (10-20 vge/cell) to avoid non-specific events and phosphorylation of target proteins was determined by immunoblot analysis. Consistent with receptor-ligand kinetics, GFR were rapidly activated within 10 minutes of treatment with ligands (e.g., GFs or HPV16), inducing concomitant phosphorylation of the downstream effector ERK1/2 (FIG. 6A). Phospho (p)-EGFR (Y1173) levels induced by HPV16 were considerably lower compared to the effect induced by EGF. The Y1173 site of EGFR is involved in MAPK signaling, and importantly, the phosphorylation levels of p-ERK1/2 induced by HPV16 were comparable to the effect of EGF (FIG. 6A).

Treatment with potent inhibitors of EGFR (PD168393), pan-FGFR inhibitor (PD173074), or general tyrosine kinase inhibitor genistein before exposure to GFs or HPV16 diminished the rapid phosphorylation of the target GFR and downstream p-ERK1/2. KGFR activation of ERK1/1 can involve EGFR crosstalk and activation, which may explain why EGFR inhibitor PD168393 blocks ERK1/2 activation by HPV16 when it also appears KGFR signaling is initiated by the virus. In contrast, daidzein, an inactive analog of genistein, elicited no inhibitory effect (FIG. 6A). To specifically query the role of EGFR activation by HPV16 , we investigated the effects of cetuximab, an EGFR-specific monoclonal antibody that binds to the EGFR extracellular domain with a higher affinity than ligands EGF or TGF-α. Cetuximab inhibits EGFR phosphorylation and activation and leads to receptor internalization and degradation. We found that cetuximab abrogated EGF- and HPV16-induced phosphorylation of EGFR and p-ERK1/2 in this assay (FIG. 6B).

GFs strongly activate ERK proteins and upon stimulation, a significant population of these kinases moves from the cytoplasm into the nucleus. P-ERK1/2-specific immunoblotting of nuclear protein fractions and confocal microscopy each revealed nuclear movement of p-ERK1/2 upon virus-induced activation (FIG. 6C, D). The timing of the p-ERK1/2 nuclear migration induced by HPV16 exposure reached its maximum after 10 minutes of exposure and indicates that signaling pathways are activated as early as five minutes post virus-host interaction. These results agree with the observation that even low-level EGFR activation can fully activate ERK1/2 in human keratinocytes.

### GFR Inhibitors Hinder HPV Infection

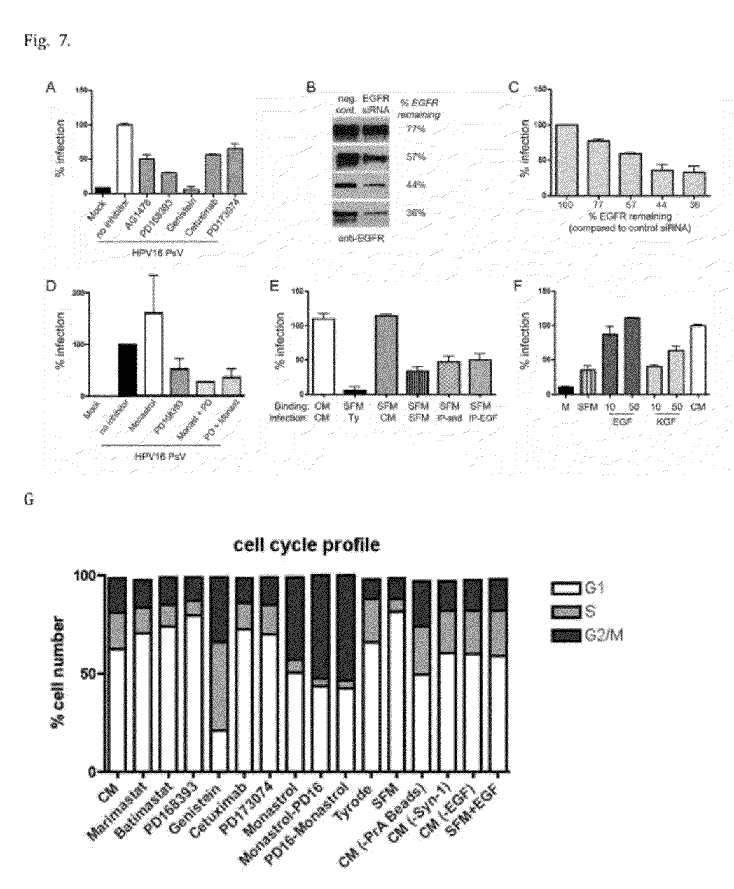

To evaluate the importance of GFR and tyrosine kinase activation in HPV infection, HaCaT cells were incubated with HPV PsV following pretreatment with and in the presence of a reversible (AG1478) or an irreversible (PD168393) EGFR-specific inhibitor, genistein, cetuximab, and the FGFR inhibitor (PD173074) in CM. Both EGFR specific biochemical inhibitors substantially blocked infection by HPV16 (>50%), while genistein almost completely blocked infection (FIG. 7A). Treatment of HaCaT cells with an EGFR blocking antibody (cetuximab) or FGFRJKGFR inhibitor (PD173074) reduced infectivity by 50 and 35%, respectively. Similar GFR signaling activation and response to inhibitors was observed with HPV31 PsV and with particles carrying the viral genome, ruling out a luciferase-specific inhibition. The complete inhibition of HPV infection by blocking receptor tyrosine kinase (RTK) signaling with genistein demonstrates the requirement for this class of receptors in HPV infection. Specific inhibitors of EGFR (e.g., cetuximab, AG1478, or PD168393) or of KGFR (PD173074), while completely abrogating signaling from their respective RTK under conditions described in FIG. 6, only partially reduced HPV infection under conditions in CM (FIG. 7A). These data show that no single RTK is essential for HPV16 infection of HaCaT keratinocytes. Rather, EGFR, KGFR, and potentially other RTK are important mediators of HPV infection.

A genetic approach using siRNA to inhibit EGFR expression gave comparable results. Typical transfection efficiency of HaCaT cells was ≈70% as monitored by fluorescein-labeled control siRNA. EGFR knockdown was assessed in four separate transfections at 48 hours post transfection by immunoblot and ranged from remaining EGFR expression of 77-36% compared to cells transfected with a nonspecific control siRNA (FIG. 7B). HPV16 infection was performed 24 hours post transfection and the level of infection 24 hours later was reduced in a dose-dependent manner that paralleled the level of EGFR knockdown (FIG. 7C).

Because progression into early M-phase is needed for HPV infection, it was important to rule out that the inhibitors blocked infection via cell cycle changes. Therefore, we assayed the fraction of cells in each phase of the cell cycle during the inhibitor treatments under which infections were determined above. In no cases did the inhibitors arrest the cells in any one cell cycle phase. Further, there was no correlation between infection inhibition and cell cycle distribution under the assay conditions employed (FIG. 7G). For example, the distribution of cells in the G1, S, or G2/M phases of the cell cycle were relatively similar whether cells were grown in CM and infected with HPV16 with no treatment or treated with batimastat, marimastat, PD173074, PD168393, or cetuximab and infected. However infection levels ranged from 0% inhibition with no inhibitor to nearly 90% inhibition with marimastat. Specifically, the moderate changes observed in the number of cells in G2/M phase were not sufficient to account for the levels of infection inhibition demonstrated for each inhibitor tested. The most striking result was found when using monastrol, which increases the number of cells in late M-phase and promotes infection. When PD168393 treatment was added with monastrol, a similar cell cycle profile was seen, yet infection was dramatically inhibited by ≈70% (FIGS. 7G and 7D, respectively). These data indicate that cell cycle effects cannot account for the inhibition of early infection events by these various compounds.

Serum Enhances HaCaT Cell Infection with HPV

The cell binding and infectivity of some viruses can be affected by medium composition. We also found HPV infection of HaCaT cells to be quite dependent upon the nature of the experimental media. Equal doses of HPV16 were allowed to attach to serum-starved cells in SFM at 4° C. and, after washing away unbound virus, cells were incubated at 37° C. overnight in Tyrode's buffer, SFM or CM. As a positive control HaCaT cells were used where virus binding and infection were both performed in the presence of CM. As shown in FIG. 7E, there was no difference in infection levels between the positive control and cells where virus was bound to cells in the presence of SFM and thereafter incubated with CM. This demonstrates that virus binding to initial attachment factors is unaffected by the nature of the media. However, there was negligible infection in cells where infection was allowed to proceed in the presence of Tyrode's buffer (−6%) and infection of cells the presence of SFM was only 38% compared to cells grown in the presence of CM (FIG. 7E). The main differences between Tyrode's buffer and SFM are amino acids and a higher glucose concentration in SFM, which has a significant effect on cell metabolism (Guertin and Sabatini, 2007). The addition of serum (containing various GFs and HSPGs) to SFM significantly increased virus infection, indicating an important role for these molecules in virus uptake and infection. As we found that CM contains considerable amounts of syndecane-1, likely in complex with GF (Harmer, 2006) (FIG. 2A), we predicted depletion of syndecane-1 from the CM would remove a substantial level of components needed for infection. As expected, when the CM was stripped of syndecane-1 by IP, infection levels were reduced to levels similar to those in SFM (FIG. 7E). Similarly, we used immunoprecipitation to deplete the serum of EGF and again, infection levels were reduced (FIG. 7E).

Based on our finding that bound HPV particles become decorated with HS and are released from cells plus the fact that main constituents of serum include albumin and GF, we tested the theory that GF facilitate movement from attachment factor to secondary receptors. If our hypothesis proves correct and GF are responsible for bridging the soluble HMW HPV-HSPG complexes to secondary receptors, then reconstituting GF in SFM should restore infectivity. Although the addition of albumin did not enhance infectivity in SFM (not shown), the addition of EGF and KGF in SFM dramatically restored infection in dose dependent manners. EGF was able to fully restore infection levels but KGF at the same concentrations was only able to partially restore infection levels to those seen in CM (FIG. 7F). Thus, we show syndecane-1 plus either EGF or KGF are required for HPV16 infection of human keratinocytes. Although infection in SFM increased the number of cells in G1 phase, the depletion conditions did not alter the cell cycle profiles significantly from that in CM or in SFM plus EGF (FIG. 7G), suggesting cell cycle changes alone could not account for infection inhibition.

### HPV Infection Model

Viruses hijack many normal cellular processes in order to gain entry into a host cell. Some viruses have multiple structural proteins that are required to initiate cellular uptake, whereas other viruses use one or two viral capsid proteins for interaction. Certain viruses may bind directly to uptake receptors, whereas others first bind to cell attachment factors that are generally thought to lack specificity before particles are laterally transferred to entry receptors. Generally implicit when referring to lateral transfer is the physical movement on the plasma membrane. In several cases, early binding events may trigger capsid conformational changes that permit movement to and/or interaction with an entry receptor, dictate signaling to initiate endocytosis, and/or activate membrane fusion activities. Although a variety of cellular interacting factors have been identified for the HPV infection process, many specifics of the early stages of HPV-cell interaction have been enigmatic. HPV particles engage HSPG attachment moieties and are thought to dissociate from HSPGs or to move laterally to interact with secondary receptors that promote endocytosis. Yet, the mechanism facilitating virus movement from primary attachment to the internalization receptor(s), or whether the process is spontaneous or highly controlled, has not been defined.

We report evidence for a novel mechanism of virus transfer from general attachment factors to secondary receptors (FIG. 8B). Although this process is unusual and complex from the viral standpoint, the procedure involves the normal systematic release of HSPG-bioactive factor-containing complexes from the cells to promote signaling via a cognate cellular receptor. We show HPV particles associate with HSPG-GF complexes that are liberated from cells as soluble effectors that then use the specificity of the GF or other bioactive molecule to interact with the cognate cellular receptor (i.e., a RTK/GF receptor) for initiation of infection. In FIG. 8B, we propose a new model for the HPV infection process that incorporates findings from the studies reported herein and that also is consistent with a number of prior studies. Our model integrates many of the more difficult to explain observations about HPV-cell interactions and entry, which are outlined below.

Mechanism of Lateral Virion Movement from Bound HSPG at the Plasma Membrane.

Most BIN types use HSPG for initial host cell attachment. Syndecan-1, the predominant HSPG in keratinocytes, is a demonstrated primary HPV-cellular interacting partner. The HPV-HS interaction was first thought to be nonspecific, but recent reports show that HS modifications by sulfate groups are essential for HPV types 11, 16 and 33 capsid interactions with cells. HPV L1 proteins mediate the capsid binding to HSPG; L1-only VLP are capable of normal cellular internalization and the L2 protein does not contribute to the initial interaction. It has been proposed that L1-HSPG binding induces conformational changes in the viral capsid that cause the normally hidden N-terminal region of L2 to become accessible to furin cleavage. This action on L2 is suggested to trigger reduced affinity of capsids for HS, with these events exposing a viral binding site for the as yet unidentified cell surface receptor involved in infectious internalization. However, to cause capsid dissociation from HSPGs, a substantial conformational change would seem to be required to alter the strong binding affinity of L1 VLP for cells (10⁻¹² M). It is difficult to imagine how cleavage of L2 could alter L1's affinity for HSPG to this extent. Moreover, no conformational changes in HPV16 capsid structure have been reported that are of the extent expected to cause capsid dissociation from HSPGs.

Our results show HPV16 is not released from syndecane-1 by dissociation, but rather in soluble complex with the HSPG ectodomain as well as various GFs, many from the EGF family. Soluble, bioactive complexes like GFs and ligands normally accumulate on HSPG ectodomains and are routinely released by proteolytic cleavage of the core HSPG-containing proteins with MMPs as well as by enzymatic cleavage of the HS chains of proteoglycans by heparinase. Consistent with this, we found HPV infection also is dependent upon the function of MMPs. Thus, HPV capsids become modified, a process we term as “decorated,” by association with these cellular factors and we demonstrate the important role of these soluble HS-GF complexes that decorate HPV particles in infection.

Differences in HSPG Dependence between Tissue-Derived and 293T System-Derived Virus Preparations.

Organotypic (raft) tissue-derived HPV31 virions infect HaCaT cells in an HSPG-independent manner, whereas HPV31 PsVs from the 293T system are HSPG-dependent in the same cells. Moreover, it is difficult to achieve a high level of purity of viral particles extracted from organotypic (raft) tissues relative to HPV particles obtained from the 293T expression system. This may be due to lower yields of virus particles per cell in the raft system compared to the 293T system. In the context of this study, it may be that raft tissue-derived virions become decorated with HS-GF during the virion isolation process and, like those decorated particles released in our co-culture system (FIG. 4), can bypass the need for HSPG association on newly exposed naïve cells. HPV particle decoration with HS-GF could occur in various ways via contributions of the cell type or tissues wherefrom viral stocks are obtained. Low-level HPV capsid decoration occurring during assembly and purification from 293T cells likely contributes to the basal levels of infection observed in the absence of HSPG or serum components (FIGS. 4G, 7E, 7F). Differences in HPV particle decoration due to isolation techniques could result in quantitatively disparate phenotypes depending upon the assays. With regard to the mode of HPV infection inhibition by heparinase, our work suggests that exogenous heparinase treatment is likely to disrupt the HPV-HS-GF complex, wherein HPV association with GF is needed to bind the GFR and initiate signals important for infection.

We cannot, however, rule out the possibility that other structural modifications with functional consequences occur differentially during virion morphogenesis in the raft tissue culture system compared to the 293T system. Nevertheless, many observations support the utility and biological relevance of PsV for functional studies. Self-assembling VLP and PsV capsids containing L1 and L2 are structurally indistinguishable from wart-derived HPV virions. L1-only HPV VLPs mimic wart-derived virions functionally such that they elicit neutralizing antibodies in vivo that have shown long-term protection from infection in animal models and in clinical trials. Indeed, these L1-only VLPs are the basis for the successful HPV vaccines in use throughout the world today.

HPV PsV expressed from capsid genes of carcinogenic HPV types like HPV16 have a number of advantages over tissue-derived virions. Virions for carcinogenic HPV types have never been purified in useful levels from human lesions. As noted above, PsV can be produced in very high titers and can be purified at much higher degrees compared to virions obtained from the organotypic (raft) tissue culture system. A careful study of xenograft tissue-derived cotton tailed rabbit PV (CRPV) virions to 293T-produced CRPV virions established that the virion stocks were essentially indistinguishable as assayed by susceptibility to antibody-mediated neutralization, papilloma induction, and gene expression within lesions in rabbits. Thus, PsV provide an accepted substitute for working with high-titer carcinogenic HPV virions.

### Long Internalization Kinetics.

Methods to determine the rate of virus penetration into target cells often employ cell surface inhibitors (e.g., neutralization of infection by extracellular acid wash, antibody, or drugs) added over a time course after virus attachment. Quantitative infection data, therefore, reflect an average time for half the population of virions to escape the extracellular environment and cause infection (entry half-time). Reported entry half times for HPVs range from four to 24 hours. Although we reported a 14 hour internalization half-time for HPV31 in HaCaT cells, we also showed HPV31 early transcripts can be detected by RT-PCR as early as four hours post infection. These observations suggest that some HPV particles are able to enter via an infectious route much more quickly than others. Based on the finding in this study and the normal biology of HS-GF complexes, we reason that the protracted and variable HPV entry timing is due to the multiple locations and ways that virions can become decorated with HS-GF complexes (FIG. 8B). Particles decorated with HS-GF during isolation or potentially associating with these soluble materials in serum (FIG. 8B(v)) may be readily able to directly engage the entry receptor, effectively bypassing the more time consuming steps of HSPG-GF interaction and subsequent enzymatic release of HMW complexes. Data showing that RTK/GFR signaling can occur minutes after virus exposure also support this idea.

The preferential association of HPV with the ECM and basement membrane appears to be due to interactions with laminin 332 (formerly named laminin 5; FIG. 8B(ii)). This is likely because laminin 332 is a depot for HS-GF complexes to which HPV can attach, and these active complexes can be liberated by heparinases and sheddases. Our co-culture assay does not differentiate between virus released from the cell surface or the ECM. The disappearance of ECM-bound HPV over time suggests that the release of ECM- and plasma membrane-bound HPV-HS-GF complexes could contribute to the infectious process. Thus, longer internalization kinetics would be expected if HPV capsids associate with HS-GF by binding HSPG on the plasma membrane, or by associating with the HS-GF complexes that are normally sequestered on the ECM or the basement membrane. MMP- or heparinase-mediated release of these HWM HPV-HS-GF complexes would be required for subsequent engagement of the entry receptor (FIG. 1B(iv)). Experimentally, the varied means and locations of HPV association with HS-GF complexes, the time needed for ectodomain release and subsequent secondary receptor interaction, and formation of the endocytic signals and machinery would logically give rise to a variable and protracted time course of virus binding to the internalization receptor and endocytic uptake.

### Many Signal Cascades are Activated.

HPV-cell interactions activate a number of signal cascades that can induce mitosis and lead to cell proliferation. PV VLPs were found to activate the Ras/MAPK pathway with maximal ERK1/2 phosphorylation 30 minutes post exposure. This and PI3K signaling were attributed to VLP interaction with the alpha-6 beta-4 integrin. FAK signaling via α6 integrin occurs as early as five minutes post HPV16 PsV exposure. Consistent with finding HPV16 in HMW soluble HS-GF complexes, we show the virus associates with GFRs and activates signaling cascades that are essential to the infection process in human keratinocytes. Interestingly, cross talk between integrins and RTK-like EGFR influence FAK in its role triggering several pathways leading to ERK activation. Although integrin-mediated activation of EGFR can occur in a ligand-independent manner, our results using specific GFR ligands (e.g., EGF, KGF), ligand-blocking antibody, and several kinase inhibitors show that GFR ligands contribute significantly to the infectious process of HPV16 in HaCaT cells. Association of EGFR with α6β4 integrin and EGF-induced phosphorylation of β4 integrin play roles in a wounded environment, an important mediator of HPV infection as discussed below.

The experimental design to detect signal-related phospho-proteins upon virus exposure in FIG. 6 reflects those HPV virions that can interact quickly with the RTK. Interaction of bioactive HS-GF with their specific receptors leads to the generation of cellular signaling pathways that promote their internalization by endocytosis. Thus, we infer that these HPV-GFR interactions promote the internalization of HPV. None of the inhibitors used herein have been shown to prevent ligand internalization. Thus, the HPV internalization that occurs during GFR inhibition must primarily shunt HPV into a nonproductive pathway. Together our findings support a model in which HPV decorated with HS-GF complexes is released from cells to engage GFR and subsequently activate viral entry (FIG. 8B). In addition, RTK signaling may play an indirect role in virus endocytosis.

### Difficulty in Identifying a Single Entry Receptor and Disparate Entry Routes Identified.

Integrins, laminin 332, and syndecans have all been shown to interact with HPVs. Each of these interactions may be due to the association of HPV particles with HSPGs, which are direct modifiers of syndecane-1 and interaction partners with laminin 332 and α6 integrin (FIG. 8). We demonstrated HPVs associate with HS containing various GFs and subsequently interact with EGFR and KGFR, further illustrating that HPV infection involves multiple pathways. HPV16 has been shown to enter cells via clathrin-dependent and clathrin-/caveolin-independent pathways. Our model showing the importance of EGFR and KGFR pathways explains how different receptors can be engaged under differing circumstances. EGFR and KGFR internalization are typically clathrin-dependent. However, EGFR entry can also involve slower clathrin-independent modes and EGFR associates with lipid microdomains when coupled with alpha-6 beta-4 integrin. Blocking ligand binding or the kinase activity of these receptors with specific inhibitors clearly shows their significant roles in HPV infection. CHO cells lack EGFR ErbBl, but are readily infected with HPVs, further demonstrating the ability of HPV to utilize multiple routes of infection. Similarly, Vaccinia virus infection of HeLa cells is EGFR dependent, but the virus can infect CHO cells using alternative mechanism. These observations explain why it is difficult to completely block infection targeting a single receptor or to clearly clearly a single entry pathway under varied experimental conditions.

Implications for in vivo Infections in a Wounded Environment.

Epithelial wounding, a mediator of PV infections in vivo, can lead to the influx and activation of many cell factors shown to interact with HPVs, including those we have identified in this work. GFs, cytokines, and chemokines are known mediators of wound repair. EGF and FGF-7 (KGF) are released from cells, and heightened MMP activity causes an increase in HB-EGF shedding. EGF and cytokines are involved in the regulation of syndecan shedding and KGF induces strong syndecane-1 expression beneath the basement membrane. Further, syndecane-1 expression is strongly upregulated in migrating and proliferating keratinocytes. EGFR expression transiently increases after wounding and KGFR is upregrulated at the wound margin. α6β4 integrin, a component of hemidesmosomes, performs adhesive functions by binding to laminin 332 in the basement membrane. Association of EGFR with α6β4 integrin and EGF-induced phosphorylation of beta-4 integrin is important for this disassembly of hemidesmosomes to promote cytokinesis and epithelial migration a wound-healing response. Syndecan-1 and syndecan-4 ectodomains are found in acute dermal wound fluids, where they, in turn, regulate GF activity, specifically the formation of HS-KGF complexes and actions of MMPs on shedding of EGFR ligands. Taken together, our work illustrates additional means by which HPV has adapted to utilize the environment created during wounding, which not only allows the virus access to mitotically active basal cells, but provides factors essential for the virus to infect cells with the boost of mitogenic signals.

We demonstrate the signal pathways initiated by KGFR and EGFR engagement by HPV can robustly activate the ERK1/2 pathway. An ultimate target of these mitogenic signals are the AP1 transcription factors, c-fos and c-jun, which are important for HPV early transcription and are thought to dictate the strict epithelial tropism demonstrated by HPVs. In this way, HPV interaction at the cell surface, like that of many other viruses, primes the host cell for viral gene expression and the establishment of infection.

In a broader sense, it is of particular interest to note that syndecans and other HSPG are bound by pathogens in addition to HPV, including some retroviruses, herpesviruses, flaviviruses, and bacteria like Chlamydia and Neisseria. Furthermore, post-attachment release of bound retroviruses, previously reported and ascribed to dissociation, instead may be subject a manifestation of the mechanism of cellular liberation we describe herein. This raises an exciting possibility that these retroviruses or other pathogens might also employ a soluble virus-HS-GF mode of infection under certain circumstances. Our study provides new insights into the transmission of a significant viral pathogen and reveals novel means whereby pathogens may hijack normal cell functions during infection of their hosts. Likewise, this work uncovers new targets for prophylaxis of HPV, and potentially other pathogen infections.

Thus, in one aspect, the invention provides a composition that generally includes an infection antagonist that inhibits formation of a heparin sulfate proteoglycan (HSPG)-containing infection complex. In various embodiments, the infection antagonist can include, for example, a heparanase antagonist, a sheddase antagonist, an inhibitor of an ADAM, an inhibitor of an MMP, an inhibitor of a TIMP, an inhibitor of the release of a growth factor or cytokine, an inhibitor of a molecular association involving HSPG, an inhibitor of molecular associations involving a growth factor receptor (GFR), an antagonist of a sheddase activator, an antagonist of a matrix metalloproteinase (MMP), an inhibitor of a secretase, an antagonist of growth factor-growth factor receptor (GF-GFR) binding, an antagonist of cytokine-receptor binding, an antagonist of GFR signaling, or an antagonist of receptor-mediated endocytosis.

The exemplary classes of infection antagonists identified immediately above share a common feature in that each is capable, albeit through different mechanism, of interfering with the formation of an HSPG-containing infection complex as illustrated in FIG. 8. Moreover, individual members of each class of infection antagonists are known and characterized.

In some embodiments, the composition can include a combination of two or more infection antagonists. When the composition includes a plurality of infection antagonists, each infection antagonist may be different than each of the other infection antagonists. Alternatively, a composition that includes a plurality of infection antagonists may include two or more infection antagonists from one class—e.g., antagonists of GF-GFR binding.

The composition described herein may be formulated in a composition along with a “carrier.” As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions.

By “pharmaceutically acceptable” is meant a material that is not biologically or otherwise undesirable, i.e., the material may be administered to an individual along with an infection antagonist without causing any undesirable biological effects or interacting in a deleterious manner with any of the other components of the pharmaceutical composition in which it is contained.

An infection antagonist may be formulated into a pharmaceutical composition. The pharmaceutical composition may be formulated in a variety of forms adapted to a preferred route of administration. Thus, a composition can be administered via known routes including, for example, oral, parenteral (e.g., intradermal, transcutaneous, subcutaneous, intramuscular, intravenous, intraperitoneal, etc.), or topical (e.g., intranasal, intrapulmonary, intramammary, intravaginal, intrauterine, intradermal, transcutaneous, rectally, etc.). It is foreseen that a composition can be administered to a mucosal surface, such as by administration to, for example, the nasal, vaginal, rectal, or respiratory mucosa (e.g., by spray, aerosol, or suppository). A composition also can be administered via a sustained or delayed release.

A formulation may be conveniently presented in unit dosage form and may be prepared by methods well known in the art of pharmacy. Methods of preparing a composition with a pharmaceutically acceptable carrier include the step of bringing an infection antagonist into association with a carrier that constitutes one or more accessory ingredients. In general, a formulation may be prepared by uniformly and/or intimately bringing the active compound into association with a liquid carrier, a finely divided solid carrier, or both, and then, if necessary, shaping the product into the desired for umlations.

An infection antagonist may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant, a skin penetration enhancer, a colorant, a fragrance, a flavoring, a moisturizer, a thickener, and the like.

Thus, in another aspect, the invention provides a method that generally includes administering to a subject an amount of a composition as described above effective to inhibit infection by a microorganism that that infects a host through interactions that involve HSPG.

The amount of an infection antagonist administered can vary depending on various factors including, but not limited to, the specific infection antagonist, the weight, physical condition, and/or age of the subject, and/or the route of administration. Thus, the absolute weight of an infection antagonist included in a given unit dosage form can vary widely, and depends upon factors such as the species, age, weight and physical condition of the subject, as well as the method of administration. Accordingly, it is not practical to set forth generally the amount that constitutes an amount of an infection antagonist effective for all possible applications. Those of ordinary skill in the art, however, can readily determine the appropriate amount with due consideration of such factors.

In some embodiments, the method can include administering sufficient infection antagonist to provide a dose of, for example, from about 100 ng/kg to about 50 mg/kg to the subject, although in some embodiments the methods may be performed by administering an infection antagonist in a dose outside this range. In some of these embodiments, the method includes administering sufficient infection antagonist to provide a dose of from about 10 μg/kg to about 5 mg/kg to the subject, for example, a dose of from about 100 μg/kg to about 1 mg/kg.

Alternatively, the dose may be calculated using actual body weight obtained just prior to the beginning of a treatment course. For the dosages calculated in this way, body surface area (m²) is calculated prior to the beginning of the treatment course using the Dubois method: m²=(wt kg^{0.425}×height cm^{0.725})×0.007184.

In some embodiments, the method can include administering sufficient infection antagonist to provide a dose of, for example, from about 0.01 mg/m² to about 10 mg/m².

In embodiments in which the composition includes two or more infection antagonists, the dosages described above may refer to the cumulative amount of all infection antagonists in the composition. In other embodiments, the dosages described above may refer to the amount of any individual infection antagonist.

In some embodiments, an infection antagonist may be administered, for example, from a single dose to multiple doses per week, although in some embodiments the method can be performed by administering an infection antagonist at a frequency outside this range. In certain embodiments, an infection antagonist may be administered from a once-off single dose to once per year.

In some embodiments, the microorganism can include a virus. Exemplary viruses include members of the Papillomaviridae family such as, for example, human papillomavirus (HPV); a member of the family Herpesviridae such as, for example, human herpesvirus (HHV) such as, for example, herpes simplex virus-1 (HSV-1, MTV-1), herpes simplex virus-2 (HSV-2, HHV-2), varicella zoster virus (VZV, HHV-3), Epstein-Barr virus (EBV, HHV-4), cytomegalovirus (CMV, HHV-5), roseolovirus (Herpes lymphotrophic virus, HHV-6), roseolovirus (HHV-7), or Kaposi's sarcoma-associated herpesvirus (KSHV, HHV-8); a member of the family Poxviridae such as, for example, a member of the genus Orthopoxvirus such as, for example, Vaccinia virus; or a member of the Retroviridae family such as, for example, a lentivirus such as, for example, Human Immunodeficiency Virus (HIV); or a member of the Flaviviridae family such as, for example, a virus such as, for example, hepatitis C virus (HCV) .

In other embodiments, the infection antagonist can include a bacterium.

Exemplary bacteria include members of the family Chlamydiaceae such as, for example, a member of the genus Chlamydia; members of the family Rickettsiaceae such as, for example, a member of the genus Orientia; members of the family Listeriaceae such as, for example, a member of the genus Listeria; members of the family Streptococcaceae such as, for example, a member of the genus Streptococcus; members of the family Staphylococcaceae such as, for example, a member of the genus Staphylococcus; or members of the family Neisseriaceae such as, for example, a member of the genus Neisseria.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Cell Culture, Virus Production, Infections

HaCaT cells are a spontaneously immortalized epithelial line derived from normal adult skin. HaCaT cells were maintained in DMEM/Ham's F-12 medium (Irvine Scientific, Santa Ana, Calif.), supplemented with 10% FBS (Invitrogen Corp., Carlsbad, Calif.), 4× amino acids (Invitrogen Corp., Carlsbad, Calif.), and glutamine-penicillin-streptomycin (Invitrogen Corp., Carlsbad, Calif.). CHO-K1 cells and their derivative pgsd-677 were grown in Ham's F12 medium supplemented with 10% fetal bovine serum and 1% Glutamax (Invitrogen Corp., Carlsbad, Calif.). HEK-293TT cells are derived from a human embryonic kidney cell line immortalized with SV40 large T antigen and were maintained in DMEM high glucose (Irvine Scientific, Santa Ana, Calif.) supplemented with 10% FBS, nystatin/gentamycin (Invitrogen Corp., Carlsbad, Calif.), and 0.4 μg/ml hygromycin B (Invitrogen Corp., Carlsbad, Calif.). HaCaT cells were seeded in to be 60-80% confluent on the day of infection. PsV stocks were sonicated for 30 s, added to cells (200-300 vge/cell), and incubated at 4° C. for one hour with gentle rocking to permit viral attachment. Formula were aspirated, cells were washed three times with complete medium and fresh media were added. Infections were allowed to proceed at 37° C. for 20-24 hours. After washing with PBS, cells were lysed with Promega Corp., Madison, Wis. Luciferase lysis buffer for 10 minutes at RT. The extracts were centrifuged 30 seconds at 14,000×g, and luciferase activities were measured by using the Luciferase kit assay (Promega Corp., Madison, Wis.) and a Lumat LB 9501 luminometer (Berthold Technologies U.S.A., LLC, Oak Ridge, Tenn.). Raw data were normalized by protein content using Bio-Rad protein assay. Control infections were set to 100% infection with the averages of 3-4 replicate experiments and error bars represent standard error of the mean.

Co-Culture and HPV Release Infection Assay

Donor cells were grown in monolayers up to 50% saturation on 8 mm glass coverslips in separate plates and incubated with ˜2000 vge/cell of HPV16 PsV for one hour at 4° C. (FIG. 4A). Unbound virus was washed out three times with medium, and coverslips transferred to 74-μm mesh Netwell inserts (Corning Inc., Lowell, Mass.). The PsV-exposed donor cell inserts were suspended above a subconfluent recipient cell monolayer grown in 12-well plates (FIG. 4C). After incubation of cells at 37° C. for 24 hours with gentle rocking, infection was measured in donor and recipient cells by luciferase assay. Each condition was performed in quadruplicate. Control infections were set to 100% infection with the averages of replicate experiments and error bars represent standard error of the mean.

HPV Pseudovirion (PsV) Production and Purification

HPV PsV were generated in 293TT cells as described (Campos, S. K., and M. A. Ozbun, 2009, PLoS ONE 4:e4463.; Smith, J. L., Campos, S. K., Ozbun, M. A., 2007, J Virol 81, 9922-9931). The transfection-based method for papillomavirus production was modified from that previously published (Buck, C. B., Cheng, N., Thompson, C. D., Lowy, D. R., Steven, A. C., Schiller, J. T., Trus, B. L., 2008, J. Virol. 82, 5190-5197; Buck, C. B., Pastrana, D. V., Lowy, D. R., Schiller, J. T., 2005, in: Davy, C., Doorbar, J. (Eds.), Human Papilloma Viruses: Methods and Protocols. Humana Press, Inc., Totowa, N.J., pp. 447-464.). 293TT cells were transfected by the calcium phosphate method with a luciferase reporter (pGL3-control, Promega Corp., Madison, Wis.) and either a codon-optimized HPV31-L1/ L2-expressing plasmid or a codon-optimized HPV16-L1/L2-expressing plasmid, pXULL. At 48 hours post-transfection, cells were tryspinized, pelleted, and resuspended at 1×10⁸ cells/ml in Dulbecco's phosphate-buffered saline (PBS)—9.5 mM MgCl2. Cells were lysed with 0.35% Brij58 and subjected to three freeze-thaw cycles. Unpackaged DNA was digested with 20 U/ml exonuclease V (plasmid-safe, Epicentre Biotechnologies, Madison, Wis.) and 0.3% Benzonase (Sigma-Alrich, St. Louis, Mo.). Lysates were allowed to mature overnight and then clarified by low-speed centrifugation. Supernatants were layered atop a 1.25-g/ml to 1.4-g/ml step CsCl gradient. Following centrifugation at 20,000×g for 16 to 18 hours, the viral band was extracted by side puncture. Virions were washed and concentrated in HSB (25 mM HEPES pH 7.5, 0.5 M NaCl, 1 mM MgCl₂) using Amicon Ultra-4 100,000 MWCO centrifugation filter units (Millipore Corp., Billerica, Mass.). SDS-PAGE and Coomassie Brilliant Blue staining were used to determine virion stock purity and L1 protein content. “Viral genome equivalent” (vge) titers of packaged reporter plasmids were determined by dot blot hybridization as previously described (Ozbun, M. A., 2002, J Gen Virol 83, 2753-2763; Patterson, N. A., Smith, J. L., Ozbun, M. A., 2005, J Virol 79, 6838-6847). 293TT cells, HaCaT cells, CHO-K1 cells and derivative pgsd-677 were maintained as reported (Boukamp, P., Petrussevska, R. T., Breitkreutz, D., Hornung, J., Markham, A., Fusenig, N. E., 1988, J. Cell Biol. 106, 761-771; Buck, C. B., Cheng, N., Thompson, C. D., Lowy, D. R., Steven, A. C., Schiller, J. T., Trus, B. L., 2008, J. Virol. 82, 5190-5197; Esko, J. D., Stewart, T. E., Taylor, W. H., 1985, Proc. Natl. Acad. Sci. U.S.A. 82, 3193-3201; Ozbun, M. A., 2002, J. Virol. 76, 11291-11300). HPV PsV encapsidating a luciferase reporter plasmid were generated via transfection in 293TT cells. CsCl gradient-purified PsV stocks were sonicated, added to cells in various media and incubated at 4° C. for one hour to permit viral attachment. Inocula were aspirated, cells were extensively washed, and fresh culture media or buffers were added. Infections were allowed to proceed at 37° C., typically for 24 hours before luciferase quantification. Raw data were normalized to total protein content. For the co-culture viral release assay, subconfluent donor cells grown on cover slips were incubated with PsV at 2000 vge/cell for 1 h, 4° C. (FIG. 4A). Cells were washed three times to remove unbound PsV, and coverslips transferred to 74-μm mesh plate inserts. The PsV-exposed donor cell inserts were suspended above a subconfluent recipient cell monolayer with media covering both cultures (FIG. 4C). Donor and recipient cell infections were measured by luciferase assay after incubation at 37° C. for 24 hours with gentle rocking.

### Effect of Inhibitors on HPV Infection

Subconfluent HaCaT cells were pre-treated 45-60 minutes with 1 μM AG1478 (Calbiochem, Merck KGaA, Darmstadt, Germany), 100 nM PD168393 (Calbiochem, Merck KGaA, Darmstadt, Germany), 100 μM genistein (Sigma-Aldrich, St. Louis, Mo.), 100 μM daidzein (Sigma-Aldrich, St. Louis, Mo.), 1 μM PD173074 (Calbiochem, Merck KGaA, Darmstadt, Germany), or 100-600 nM cetuximab (ImClone LLC, Bridgewater, N.J.). Cells were exposed to HPV16 or HPV31 PsV at 100 vge/cell for one hour at 4° C., then shifted to 37° C. in the presence of inhibitors for 24 hours at which time they were analyzed for luciferase expression.

### Sepharose 4B Gel Chromatography and Analysis of HMW Complexes

HaCaT cells were incubated with 200 vge/cell of HPV PsVs for 1 h at 4° C., washed three ties with media and incubated at 37° C. for various times before harvesting the experimental media. Media were subjected to low speed centrifugation to remove debris and the supernatant was concentrated on by Amicon Ultra 30K filtration (Millipore Corp., Billerica, Mass.). Sepharose 4B columns were preliminary calibrated with standard proteins as described (Cinek, T., Horejsi, V., 1992, J Immunol 149, 2262-2270). Concentrated samples were fractionated on a 1 ml Sepharose 4B column that had been washed with PBS; 0.1 ml of the sample was applied at the top and left to enter the gel for three minutes; the 0.1 ml of the eluate was collected as fraction 1. Next, 0.1 ml of PBS was applied, and fraction 2 collected in three minutes and so on. The void volume fraction of this gel (fraction 4) contains large complexes or particles (>10⁷ Da); maxima of MW standards IgM and IgG elute in fractions 8 and 10, respectively. The mini-columns were used in order to minimize the time necessary for separation; in preliminary experiments it was found that the quality of separation was comparable to that obtained with 25 ml columns. The eluted fractions were analyzed by SDS-PAGE followed by immunoblotting for HPV16 L1, proteoglycans and growth factors. The primary antibodies included anti-HPV16 L1 monoclonal (Abeam plc, Cambridge, Mass.), anti HB-EGF, anti amphiregulin (Santa Cruz Biotechnology, Inc., Santa Cruz, Calif.), anti-EGF (ProSpec-Tany TechnoGene, BioNovus Pty Ltd, Cherrybrook, NSW, Australia) and anti-heparan sulfate (Millipore Corp., Billerica, Mass.).

### Gelatin Zymography

HaCaT cells were incubated overnight following exposure to HPV PsV (100 vge/cell), culture supernatant was removed, cleared by centrifugation and concentrated by Amicon filtration. Concentrate was mixed with 6× non-reducing sample buffer and electrophoresed through a 10% acrylamide gelatin gel and analyzed as reported (Woessner, J. F. J., 1995, Methods Enymol. 248, 510-528).

### Fluorescent Staining

HaCaT cells were seeded onto glass cover slips in a 12-well plate and cultured overnight. The media were removed, and the slides were incubated with Tyrode's buffer (10 mM HEPES pH 7.4, 130 mM NaCl, 5 mM KCl, 1.4 mM CaC;₂, 1 mM MgCl₂, 5.6 mM glucose, and 0.1% BSA). After two hours of starvation, cells were incubated with HPV PsV at 4° C. After 45 minutes of incubation in wells Alexa 488-EGF (Invitrogen Corp., Carlsbad, Calif.) was added and incubated an additional 15 minutes. Unbound EGF and virus were washed out with cold Tyrode's solution and cells fixed with 4% paraformaldehyde for 45 minutes at RT. After several washes with PBS, cells were blocked with 1% BSA containing Tyrode's solution for one hour at RT, and incubated with rabbit polyclonal antibody (1:200) against HPV16 or RPV31 for one hour at RT (antibody raised to pure VLPs made in our lab, and affinity purified on Protein A column). Following several washes with PBS, slides were incubated with DyLight 594-conjugated affinityPure Donkey Anti-rabbit IgG (1:200; Jackson Immunochemicals, West Grove, PA) for 45 minutes at RT. Cells were washed again with PBS (5×, 10 minutes each) and coverslips were inverted onto Prolong Gold mounting solution. Alternatively, for visualization of KGFR and HPV co-localization, BSA-blocked cells were incubated with Bek(C8) mouse mAb (Santa Cruz Biotechnology, Inc., Santa Cruz, Calif.) and rabbit polyclonal anti-HPV16 or anti-HPV31 VLP for one hour at RT (primary antibodies diluted 1:100 in 1% BSA/Tyrode's buffer). After washing with PBS, slides were incubated with donkey anti-mouse-AF549 and donkey anti-rabbit-AF488 IgG secondary antibodies (1:200 dilution in 1% BSA/Tyrode's; both Jackson Immunochemicals, West Grove, Pa.). For detection of intracellular ERK, fixed cells were permeabilized with 0.1% TX100 containing Tyrode's buffer for five minutes. Anti p-44/42 MAPK rabbit mAb (1:200; Cell Signaling Technology, Inc., Beverly, Mass.) was used as primary antibody and goat anti rabbit Cy3 (Jackson Immunochemicals, West Grove, Pa.) was used as a secondary antibody. Vectashield mounting medium with DAPI (H-1200; Vector Laboratories, Inc., Burlingame, Calif.) was used instead of Prolong Gold. All images were acquired with a Zeiss LSM 510 META confocal system using appropriate filters. Parameters of lasers intensities were kept constant during the imaging. 3D (full projection) cell imaged were generated with Zen 2009 software (Carl Zeiss Inc., Jena, Germany), using Z-stack confocal series.

### Immunoprecipitations and Depletions

Confluent HaCaT cells were seeded as donor cells and incubated with 500 vge/cell HPV16 PsV at 4° C. for one hour as in FIG. 4A with anti-HPV16 L1 mouse mAb attached to Dynabeads—Protein A in the lower chamber (instead of recipient cells as in FIG. 4C). After two or 20 hours of incubation, beads were collected, washed several times with PBS, and solubilized in non-reducing sample buffer. Syndecan-1 was detected by immunoblot with anti-Snd rabbit serum (Millipore Corp., Billerica, Mass.). To Co-IP HPV16 and GFR, confluent HaCaT cells were incubated with 500 vge/cell HPV16 PsV at 4° C. for one hour; a second plate at the same confluence was left unexposed to PsV (for negative control). Plates were then washed twice with cold PBS and cells were solubilized with 2 ml/plate of cold lysis buffer (1% TX100, 50 mM Tris-HCl, pH 7.5, 150 mM NaCl, 1 mM EDTA, 1 mM PMSF, 10 ng/ml leupeptin, 10 ng/ml aprotinin). Insoluble materials were removed by centrifugation at 13,000 rpm for 10 minutes and supernatants were immunoprecipitated with affinity-purified rabbit polyclonal anti-HPV16 VLP antisera attached to protein A-magnetic Dynabeads (Invitrogen Corp., Carlsbad, Calif. Dynal) for one hour at 4° C. Beads were washed twice with cold lysis buffer, twice with cold PBS, solubilized in 15 ml Laemmli sample buffer and boiled for five minutes. Soluble proteins were resolved by 10% SDS-PAGE and were electro-blotted onto PVDF membranes. Membranes were probed with anti-syndecan-1 mAb (Santa Cruz Biotechnology, Inc., Santa Cruz, Calif.), anti-EGFR, and p-FGFR antibodies (Cell Signaling Technology, Inc., Beverly, Mass.). A 1:1000 dilution of rabbit TrueBlot HRP-conjugated anti-rabbit IgG (eBioscience, Inc., San Diego, Calif.) in TBS plus 0.05% Tween 20 and 1% BSA was used as a secondary antibody.

To prepare syndecan-1 and EGF-depleted medium, 20 μg anti-syndecan-1 mAb or anti-EGF mAb (Santa Cruz Biotechnology, Inc., Santa Cruz, Calif.) attached to Protein G Sepharose beads (GE Healthcare, Piscataway, N.J.) was washed with PBS and incubated with 3 ml of 10% FCS-DMEM for 3 h at RT. The suspension was filtered with 0.2 μm filter (NALGENE, Nalge Nunc International Corp., Rochester, N.Y.). 10% FCS-DMEM incubated with Protein G Sepharose beads was used as a negative control.

### EGFR and KGFR Signal Activation

Subconfluent HaCaT cells were serum-starved for 3-4 hours in Tyrode's buffer containing 0.05% BSA. After adding ˜100 vge/cell HPV16 PsV, 10 ng/ml EGF or 10 ng/ml KFG, cells were incubated at 37° C. for 10 minutes before transferring to ice and solubilizing cells with RIPA buffer. In some experiments cells were incubated with various inhibitors in Tyrode's buffer for 45 minutes and after Tyrode's washes, were incubated with virus as above in the presence of inhibitors. Lysates were clarified, mixed with Laemmli buffer and boiled for five minutes prior to SDS-PAGE Tmmunoblot was performed with various monoclonal and polyclonal antibodies: p-EGFR, p-KGFR (p-FGFR2b), p-ERK, actin. Nuclear fractionation was performed for detection of p-ERK movement into the nucleus. HaCaT cells were starved 4 h in Tyrode's solution containing 0.05% BSA, then exposed to HPV or EGF for various times. Cells were solubilized with NP40 lysis buffer and centrifuged. The pellet was incubated with nuclear extraction buffer. Following incubation on ice for one hour, the extract was clarified and the supernatant subjected to SDS-PAGE and immunoblot for analysis of p-ERK content.

It is noted that, as used in this specification and the appended claims, the singular forms “a,” “an,” and “the,” include plural referents unless expressly and unequivocally limited to one referent. Thus, for example, reference to “a virus” includes two or more different viruses. As used herein, the term “include” and its grammatical variants are intended to be non-limiting, such that recitation of items in a list is not to the exclusion of other like items that can be substituted or other items that can be added to the listed items.

Upon studying the disclosure, it will be apparent to those skilled in the art that various modifications and variations can be made in the devices and methods of various embodiments of the invention. Other embodiments will be apparent to those skilled in the art from consideration of the specification and practice of the embodiments disclosed herein. It is intended that the specification and examples be considered as examples only. The various embodiments are not necessarily mutually exclusive, as some embodiments can be combined with one or more other embodiments to form new embodiments.

What is claimed is:

1. A composition comprising:
an infection antagonist that inhibits formation of a heparan-sulfonated proteoglycan (HSPG)-con ining infection complex.

2. The composition of claim 1 wherein the infection antagonist comprises a heparanase antagonist.

3. The composition of claim 1 wherein the infection antagonist comprises a sheddase antagonist.

4. The composition of claim 1 wherein the infection antagonist comprises an inhibitor of the release of a growth factor.

5. The composition of claim 1 wherein the infection antagonist comprises an inhibitor of a molecular association involving HSPG.

6. The composition of claim 1 wherein the infection antagonist comprises an inhibitor of molecular associations involving a growth factor receptor (GFR).

7. The composition of claim 1 wherein the infection antagonist comprises an antagonist of a sheddase activator.

8. The composition of claim 1 wherein the infection antagonist comprises an antagonist of a matrix metalloproteinase (MMP).

9. The composition of claim 1 wherein the infection antagonist comprises an inhibitor of a secretase.

10. The composition of claim 1 wherein the infection antagonist comprises an antagonist of growth factor-growth factor receptor (GF-GFR) binding.

11. The composition of claim 1 wherein the infection antagonist comprises an antagonist of cytokine-receptor binding.

12. The composition of claim 1 wherein the infection antagonist comprises an antagonist of GFR signaling.

13. The composition of claim 1 wherein the infection antagonist comprises an antagonist of receptor-mediated endocytosis.

14. The composition of claim 1 further comprising a second infection antagonist that comprises a heparanase antagonist, a sheddase antagonist, an inhibitor of the release of a growth factor, an inhibitor of a molecular association involving HSPG, an inhibitor of molecular associations involving a GFR, an antagonist of a sheddase activator, an antagonist of a MMP, an inhibitor of a secretase, an antagonist of GF-GFR binding, an antagonist of cytokine-receptor binding, an antagonist of GFR signaling, or an antagonist of receptor endocytosis.

15. The composition of claim 1 further comprising a pharmaceutically acceptable carrier.

16. A method comprising:
administering to a subject an amount of a composition comprising an infection antagonist that inhibits formation of a heparin sulfate proteoglycan (HSPG)-containing infection complex effective to inhibit infection by a microorganism that that infects a host through interactions that involve HSPG.

17. The method of claim 16 wherein the microorganism comprises a virus.

18. The method of claim 17 wherein the virus comprises HPV, HIV, HCV, or herpes virus.

19. The method of claim 16 wherein the microorganism comprises a bacterium.

20. The method of claim 19 wherein the bacterium comprises a member of the genus Chlamydia, a member of the genus Orientia, a member of the genus Listeria, a member of the genus Streptococcus, a member of the genus Staphylococcus, or a member of the genus Neisseria.
